(19) United States
(12) Patent Application Publication
(10) Pub. No.: US 2004/0049241 A1
(43) Pub. Date: Mar. 11, 2004

(54) Distributed muscle stimulator

(75) Inventor: James M. Campos, Hayward, CA (US)

Correspondence Address: WOOD, HERRON & EVANS, LLP, 2700 CAREW TOWER, 441 VINE STREET, CINCINNATI, OH, 45202, US

(73) Assignee: Therapeutic Innovations, Inc., Crescent Springs, KY

(21) Appl. No.: 10/238,545

(22) Filed: Sep. 10, 2002

(51) Int. Cl. A61N 1/18

(52) U.S. Cl. 607/48

(57) ABSTRACT

A method, apparatus and program product selectively transmit charge of the same stimulating signal via multiple points of entry. Selective transmission of consecutive portions of the signal to different electrodes enhances signal effectiveness by decreasing incidences of repetition and charge concentration.

### FIELD OF THE INVENTION

The present invention relates generally to the field of electronic muscle stimulation, and more particularly to transcutaneous muscle development, relaxation and therapy.

### BACKGROUND OF THE INVENTION

The ability to stimulate or exercise muscle tissue is critical to the development and rehabilitation of muscle. In nature, alterations in ion channels cause the brain to generate electronic impulses or synapses. An impulse propagates along an axon to its termination on its way to initiating a muscle contraction. As such, characteristics of the impulse complement the active processes of the nervous system.

Man-made attempts to stimulate muscles often strive to emulate natural impulses, working within the confines of axon receptors. Therapists and athletes use machines that produce variations of such signals to develop and/or treat muscle tissue by inducing a series of contractile twitches that aggregate to form a contraction. Conventional signals embody a sequence of pulses or other waveforms optimized to produce such contractions. The pulses of a mechanically generated signal typically have different characteristics tailored to a particular stimulating application. For instance, a signal may incorporate preprogrammed pulses having different amplitudes, spacing and widths accounting for user sensitivity, circulation, muscle type, contractile return and other performance considerations discussed below. As such, signals may vary as a function of time and in a manner optimized to stimulate a muscle. The signal is transmitted to a user via a pair of electrodes, which are conventionally taped, strapped or otherwise attached to the skin of the user proximate a targeted muscle.

A transmitting, or negative, electrode of the pair transcutaneously applies the signal to the user. The signal propagates from the negative electrode through the muscle before exiting through a receiving, or positive, electrode that acts as an electrical drain to collect the signal. Thus, the tissues of the body effectively complete an electrical circuit comprising the two electrodes and the stimulator output for a given signal. In this manner, the signal leaving the negative electrode corresponds to the signal voltage of the positive electrode. The negative and positive electrodes thus form a synchronized pair that corresponds to each other on a one-on-one basis with the transmitted signal. Even where multiple electrode pairs are used in an application, each signal, or channel, corresponds exclusively to a respective electrode pair. Thus, for any signal traveling across a matched electrode pair at any given instant, one electrode of the pair must be positive, while the other is negative.

By virtue of the electrode pair configuration, the signal enters the body of the user at a localized point on the user defined by the surface area of the negative electrode. A muscle's response to the concentrated point of charge/signal entry is often characterized by a painful tightening of the muscle tissues closest to the entry point, known as tetany. This may in part be attributable to motor nerves nearest the negative electrode receiving a concentrated, sudden influx of charge without having the benefit of natural delivery receptors and other mechanisms that facilitate naturally induced signal passage. As the signal propagates from the negative terminal of the electrode pair, the signal disperses throughout the muscle tissue. As such, the charge associated with the less concentrated, and the dispersed signal affects motor and sensory nerves to a proportionately lesser extent. Moreover, natural receptors in the muscle may enable sensory nerves nearest the positive electrode to prepare or adjust accordingly for collection of the dispersed signal. As a consequence, most pain perceived by a user is experienced at the signal's point of entry proximate the negative electrode.

In the course of a stimulating session, aggravation of the sensory nerves at the location of the negative electrode can become more pronounced and eventually preclusive to a treatment application. That is, localized tetany may cause portions of the muscle to tighten near the electrodes, particularly those areas of the muscle proximate the negative electrode, while the areas between the electrodes becomes unresponsive to repeated signals over time. Pain originating from the sensory nerves that are affected by the signal may eventually overcome a patient. Such pain may consequently cause a user to lower signal strength below that of a useful level or end a session altogether.

This condition is exacerbated by the fixed nature of the electrode pair configuration. Though some configurations allow for either electrode to alternatively fulfill the function of the negative electrode for a given instant, a signal must still enter through a single point on the user's skin corresponding to the negative electrode at that instant. It would be disruptive and impractical to detach and reapply electrodes to different locations within the course of an application. As a consequence, the same muscle tissue proximate the negative electrode repetitively receives the brunt of the charge conveyed by repeated signals. This exposure to repeated pulses heightens pain perceived by the user near the localized point of entry frustrating treatment.

Moreover, the repetition promulgated by the stationary nature of the electrodes is experienced in varying degrees by all tissues of a muscle. For instance, the positioning of the electrode pair configuration onto the user fixes the path traveled by a repeated signal as it propagates throughout the muscle. Furthermore, the relative amount of charge received by motor and sensory nerves of a muscle remains proportionally consistent as between all nerves/tissues of the treated area. For example, a nerve located directly at a signal's point of entry must consistently process a larger portion of charge than that of the dispersed signal applied to a nerve more distanced from the electrode.

The repetitious nature of the electrode configuration translates into the same portion of a muscle receiving proportionately the same charge from repeated pulse sequences of repeated signals. In this manner, the placement and associated signal paths associated with known electrode pair applications contribute to a second layer of repetition. That is, not only does the muscle receive repeated pulse sequences, but the pulses enter and propagate throughout the muscle at relatively the same location within the muscle. Thus, the motor and sensory nerves of the muscle receive proportionally similar charge with repeated frequency over the course of a stimulating session.

Over time, such repetition can undermine therapeutic and developmental efforts. The repetitious nature of conventional signals and associated delivery mechanisms can frustrate attempts to initiate and sustain comparable contractions. That is, the repetition promotes negative performance factors that can diminish the penetrative and other effects of the signal needed for sustained stimulation. For instance, repeated pulses will increasingly stress and deplete energy supplies of muscle tissues over time. Repeated applications can further produce relatively little beneficial effect, because the muscles are being stretched out of shape, traumatized almost as much as they are being treated. Additionally, repeated signals can sting the skin of the user at the electrodes.

Known techniques used to address such factors include incorporating periods of recovery in between pulses. Sufficient lengths of such periods may allow the muscle to partially prepare for another contractile twitch. Muscle may use this short period between pulses to replenish a portion of expended ATP and calcium ions before a subsequent pulse. Each rest time between pulses also allows the body an opportunity to partially reset electrical polarities skewed by its preceding pulse by dissipating some capacitive charge retained in the skin.

Despite these provisions, known signal applications still suffer diminished returns with successive pulses applied to the same relative locations of a muscle due to nutritional depletion and motor nerve boredom.

Unless the pulse rate is so slow that it causes a painful, jerking sensation, there is typically an inadequate amount of time between pulses to allow for complete replenishment and electrical recovery. Consequently, repeated signals incrementally drain overall muscle resources.

As a muscle's strength and supply wane, so does its ability to contract. As such, a subsequent pulse applied to the same location, which is identical in polarity, amplitude, shape and timing will produce shallow contractions that result in less penetration than the preceding pulse. Less penetration translates into less muscle development, as weaker contractions fail to increase blood flow to required muscle tissue levels as needed for muscle treatment or development. Increasing voltages associated with a stimulating signal to achieve proportionally greater penetration will induce preclusive pain and tetany.

Still other obstacles associated with repetition hinder the effectiveness of conventional signal applications. Namely, accommodation may prevent repeated pulses from penetrating deeply into the muscle, mitigating the potential benefit of successive pulses. Muscle accommodation regards the ability of the body to adapt to constant and repeated stimuli. Such stimuli include the successive pulses of conventional muscle stimulators. As such, a muscle at a particular location adapts to subsequent pulses applied to the same location in such a manner as it fails to achieve the same level of potential in response to a repeated pulse. Two major factors contributing to accommodation relate to electrical polarity and nutritional supply as discussed herein.

To compensate for the detrimental effects of accommodation, some applications attempt to increase the voltage of subsequent pulses to maintain comparable levels of stimulation. However, such attempts are often frustrated by preclusive pain associated with the reaction of motor and sensory nerves proximate where the signal enters the body.

Other applications attempt to combat accommodation by varying pulse shape, width, height and frequency. Although such techniques can realize somewhat greater contractile reactions with less voltage, a targeted muscle at a given location still twitches in response to each pulse to a lesser degree than to the previous pulse at the same location. Furthermore, while marginally effective in temporarily achieving deeper penetration, such attempts still result in preclusive tetany and other pain that frustrates further treatment. In part, this pain stems from an inability of known applications and pulse variations to affect motor nerves (associated with muscle treatment and/or development) to the same degree as sensory nerves (associated with pain). Thus, conventional pulse designers are limited in the range of voltage they can apply and the depth of contractile reactions they can achieve. Moreover, such variation of signal characteristics fails to address repetitive effects borne of the electrode pair placement.

Conventional techniques further fail to uniformly address different tissues of a muscle implicated in a treatment/development session. As discussed above, the regularity of the path traveled by a signal as it propagates throughout a muscle or muscle grouping may consistently apply a large charge to tissues proximate the electrodes, while relatively neglecting tissues more distal to the signal path. An inability of prior art pulse applications to simultaneously and uniformly stimulate different muscles often results in disproportionate muscle tone and little to no development. Furthermore, the path traveled by the electrical signal between the electrodes remains static over the course of repeated applications, promoting disproportionate muscle growth, if such growth occurs at all. Such undesirable development detrimentally impacts balance, mobility and other motor functions.

Consequently, what is needed is an improved signal application capable of effectively exercising muscle tissue, while accounting for comfort, nutritional, balance and accommodation considerations.

### SUMMARY OF THE INVENTION

The invention addresses these and other problems associated with the prior art by providing in one respect an improved mechanism for stimulating a musculature in a manner that decreases incidences of repetition and charge concentration. In accordance with the principles of the present invention, at least a portion of a signal may be selectively applied to a user via any combination of a plurality of electrodes. Such control can allow two or more electrodes to convey a portion of the total charge associated with the signal over a larger surface area. That is, charge may be apportioned between multiple points of entry. The distribution of charge afforded by multiple points of entry translates into less perceived pain, as well as diminished effects from nutritional depletion. By apportioning the contact points and areas associated with the entry of the signal, no single point along a musculature has to be subjected to a concentrate charge associated with the ingressing signal. Subsequently, larger and more sustained applications of charge may be realized with less pain to realize greater and more uniform contractile reactions.

In one embodiment, each electrode may, in turn, assume the role of a positive electrode, while the remaining electrodes produce negative signals. The formerly positive electrode may then transition to negative, while one of the remaining electrodes becomes positive. In this fashion, the distributed electrode configuration can further combat problems born of repetition that plague conventional applications by interjecting an additional layer of variation. In accordance with an aspect of the present invention, consecutive portions of the signal may programmatically or otherwise be selectively transmitted to different electrodes over the course of a stimulating session. That is, a first electrode or group of electrodes may transmit the first portion of the signal during a first time period, while a subsequent portion of the signal may be selectively applied to the musculature via a second electrode or group of electrodes.

An operator may capitalize on this control to vary the geometry of signal entry with respect to its particular point of entry along a musculature. Thus, the same tissues of a musculature may experience different pulse characteristics, delivery and associated contractile effects over time. Such variation may function to minimize detrimental effects attributable to repetition. The selectivity further means a particular area of a musculature may receive portions of a signal that are particularly suited for the tissues of that area. Thus, a signal can be configured and delivered in a manner that accounts for each pulse or other portion of a signal as a function of both time and musculature composition.

By virtue of the foregoing there is thus provided an improved method, apparatus and program product for stimulating a musculature in a manner that addresses above-identified shortcomings of known systems. These and other objects and advantages of the present invention shall be made apparent from the accompanying drawings and the description thereof.

### BRIEF DESCRIPTION OF THE DRAWINGS

The accompanying drawings, which are incorporated in and constitute a part of this specification, illustrate embodiments of the invention and, together with a general description of the invention given above, and the detailed description of the embodiments given below, serve to explain the principles of the invention.

FIG. 1 illustrates a block diagram of an apparatus suited for stimulating a muscle in accordance with the principles of the present invention;

FIG. 2 shows a block diagram of another apparatus suited to stimulate a muscle in accordance with the principles of the present invention;

FIG. 3 is a graph that illustrates time slots assignable to firing sequences of the electrodes of FIGS. 1 and 2 as a function of time;

FIG. 4 shows in greater detail generating circuitry suited for implementation within the apparatus of FIG. 2;

FIG. 5 illustrates a first user interface suited for implementation within the apparatus of FIG. 2;

FIG. 6 shows a second user interface suited for implementation within the apparatus of FIG. 1 or 2; and

FIG. 7 shows a third user interface suited for implementation within the apparatus of FIG. 1 or 2 and configured to attach to clothing of a user.

### DETAILED DESCRIPTION OF SPECIFIC EMBODIMENTS

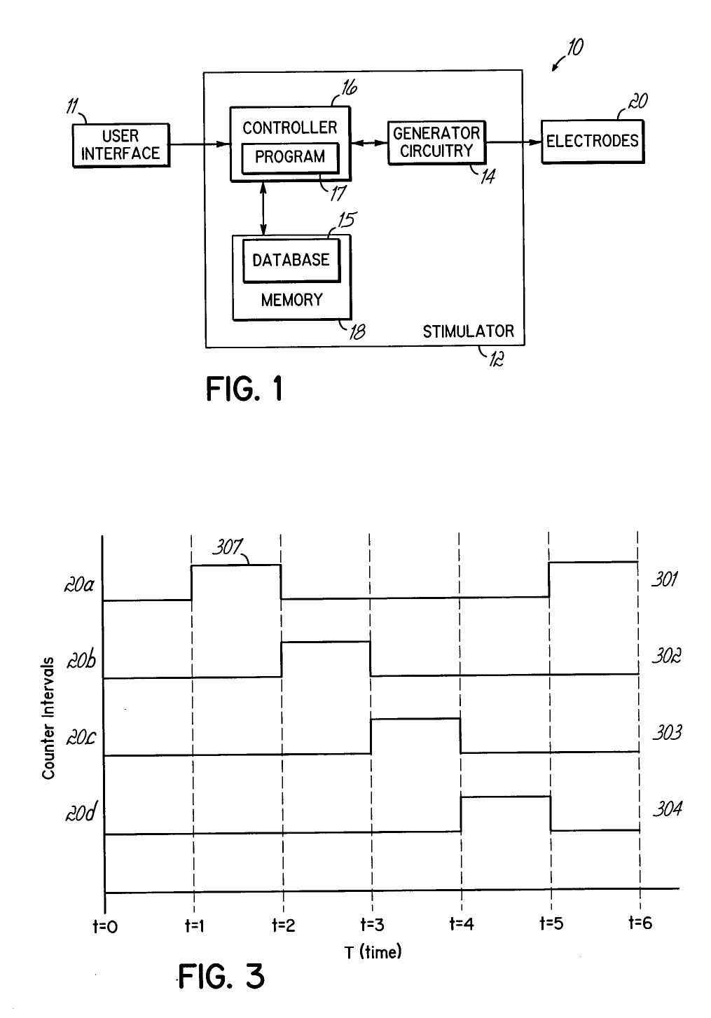

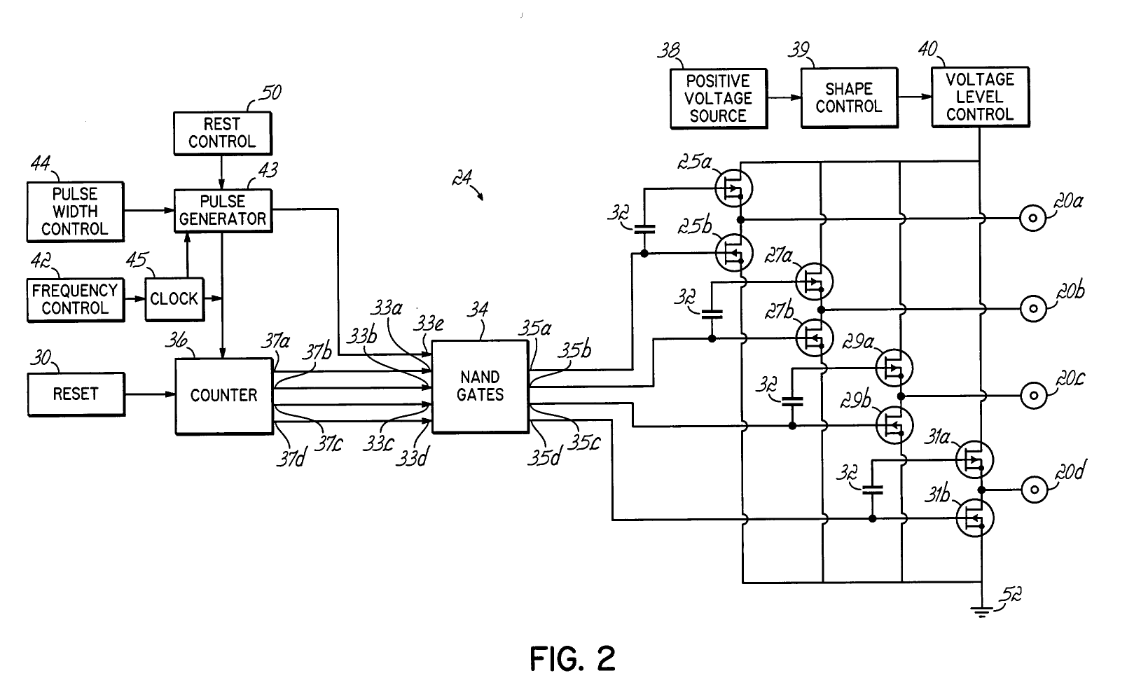

Embodiments shown in FIGS. 1 and 2 that are consistent with the principles of the present invention may apply an electronic signal to the skin of a user in order to stimulate tissues of a musculature. Significantly, charge conveyed by the signal may be selectively applied via any combination of a plurality of electrodes 20 forming multiple points of entry for the signal. As such, the charge is apportioned in that the negative side of the signal can enter the skin through more than one electrode. To this end, a plurality of negative (transmitting) electrodes 20 may be configured to have a reference ground voltage in common with each other and a positive (receiving) electrode 20 such that the electrodes 20 comprise a single circuit.

The dispersed electrode configuration may translate into less perceived pain, as well as diminished effects from accommodation, polar imbalance and nutritional depletion. To this end, an embodiment of the present invention may capitalize firstly on a phenomenon resulting in a positive electrode producing a more comfortable and secondly a more uniform and deeper contraction signal than that produced by a corresponding negative electrode. That is, the charge applied by the negative electrode of a conventional application is often characterized as inducing only shallow, localized contractions associated with tetany. In avoiding such tetany, the present embodiment not only spreads out charge from negative electrodes over a larger surface area, but it allows an operator to increase the signal by virtue of allowing the positive electrode to realize the contraction with less perceived pain. By apportioning the contact points and areas associated with the entry of the signal, pain accompanying the transmitted signal at the distributed points/electrodes is likewise apportioned. As a consequence, typically no single area on the user is subjected to a full dose of charge associated with the ingressing signal. In the absence of a sensory nerve reacting to such a concentrated charge, the normally hard and undesirable contractile effect of the negative pulse are overshadowed by the preferred contractions induced by the positive pulse. Subsequently, larger applications of charge may be employed with less pain to realize more uniform and otherwise desirable contractile reactions throughout targeted muscle groups.

The distributed electrode configuration further combats problems born of repetition that plague conventional applications by enabling an additional layer of control. Portions of a repeating signal may programmatically or otherwise be selectively transmitted to different electrodes over the course of an application(s). An operator may capitalize on this control to vary the sequence that a signal enters a musculature in relation to the point of entry of the signal. Thus, the same tissues of a musculature may experience different pulse characteristics, mitigating detrimental effects attributable to repetition.

In this manner, an embodiment of the present invention may avoid detrimental chemical and biological effects that could otherwise result under certain circumstances where the same electrode was kept positive at all times. The selectivity further means a particular area of a musculature may receive portions of a signal that are especially incorporated into the signal for the purpose of stimulating that particular area. Thus, an embodiment of the present invention allows an operator to tailor a signal according to individual areas of a musculature.

Generally, the apparatus 10 of FIG. 1 transcutaneously applies the electronic signal across a targeted musculature via the plurality of electrodes 20. For purposes of the present invention, a musculature may comprise a single muscle, as well as some muscle group, combination or chain. Of note, the plurality of electrodes typically includes three (or as drawn, four) or more electrodes 20, in that the number of electrodes 20 utilized in a given application can be in some measure proportional to comfort gains realized. An electrode can comprise any mechanism suited to deliver a charge to skin, to include metal, plastic and wireless devices/transmitters. Moreover, the principles of the present invention are nonspecific to any known signal or pulse regime. Consequently, an embodiment of the invention can accommodate and actually enhance all known prior art signal schemes and variations. For purposes of the embodiment, a signal may comprise an entire signal or a portion of a signal, such as a pulse of a signal, a portion of a pulse, a resonant harmonic, or any other increment or derivation of a stimulating signal.

FIG. 1 shows a user interface 11 coupled to a stimulator 12 and an associated generator circuitry 14. A controller 16, or suitable microprocessor, may receive input generated or statically preset from the interface 11. The stimulator 12 may use the input to configure an electrical signal operable to selectively stimulate targeted muscle groups. To this end, the stimulator 12 may correlate the user input with signal profiles stored in a database 15 resident in a memory 18. As discussed below, each profile may embody signal characteristics optimized to selectively stimulate muscle tissue with regard to its location within a musculature.

The signal characteristics are further optimized in accordance with a distributed electrode transmission configuration, which effectively divides the negative signal among at least two electrodes 20 as the electrical signal enters the musculature through the skin of a user and exits through one positive electrode.

In this manner, the musculature can be more uniformly developed or addressed for therapy, while accounting for patient and athlete balance concerns. Superior balance is achieved by virtue of an electrode receiving a large current when operating in positive mode, and a proportionately smaller current when operating in negative mode that is nonetheless sufficient to add up to a zero net charge. Associated techniques further mitigate pain associated with musculature stretching, as well as dermal sting associated with excessive current. Thus, cohesion between the electrical signal and distributed method of delivery can translate into deeper muscle penetration in the face of accommodation, polarization and nutritional depletion. Moreover, the apportioned charge upon entry means the charge does not overwhelm any single sensory nerve of the user, causing less perceived discomfort.

Regarding the characteristics of the electrical signal, the controller 16 may process information extracted from the database 15 for the purpose of sending a command to the generating circuitry 14. As discussed below in greater detail, the command may reflect information extracted from the database 1 5, to include stored profiles conveying electrode/signal firing assignments. Such assignment information may instruct the circuitry 1 4 as to which of the electrodes 20 will actively transmit a portion of the signal. The profiles may further account for what portions of the signal should go to specified electrode locations along the musculature as a function of time.

In response to a command from the stimulator 1 2 conveying parameters specific to a signal, the generating circuitry 1 4 may create and transmit a signal to the user via at least two of the electrodes 20. Of note, a transmission medium suited to convey the signals may comprise multiple cables or cordless circuits, depending on how many channels are conveyed by the generator to the electrodes 20. As such, the parameters of the generated signal will correspond to those indicated by user input. For instance, the width, amplitude, frequency and shape of each pulse of the electrical signal can be selectively modified to achieve a desired effect. As shown in FIG. 1 and discussed below in detail, the generator circuitry 1 4 may produce additional, complimentary signals for application to additional electrodes 20.

The electrodes 20 of FIG. 1 may contact the skin of a user proximate a musculature to be exercised, relaxed or otherwise treated. At least three electrodes, and typically four or more can be positioned around the extremities of the musculature. As such, applied signals may propagate inwardly from the ends of the musculature towards its center prior to exiting through one or more electrodes 20. Of note, this feature of the apparatus 1 0 permits relatively distant muscle groups to be exercised as the generated signal propagates throughout the body from the electrodes 20. Another embodiment may include a fifth electrode centered directly over a targeted musculature to provide more focused signal travel and associated stimulation.

FIG. 2 shows another block diagram having circuitry 24 suited to generate and transmit distributed signals in accordance with the principals of the present invention. The exemplary circuitry 24 of FIG. 2 includes four electrodes 20a-d. Each electrode is typically spaced on the skin of a user proximate a targeted musculature. As discussed above, however, other applications may incorporate fewer or larger numbers of electrodes, or irregularly spaced configurations. Such variation may realize greater diffusion of pain associated with the voltage entering the musculature. Electrodes may be peripherally positioned around a particular tissue of a musculature in which an operator wishes to stimulate blood flow. The spacing and other positioning of electrodes may reflect a signal protocol/profile that accounts for transmission of the signal to a selected electrode as a function of time. As noted above, another configuration may include four electrodes positioned around the periphery of a musculature, with a fifth electrode transcutaneously attached proximate the center of the musculature.

Moreover, one skilled in the art will appreciate that there are a number of different ways in which the functionality of the circuitry 24 can be realized. That is, any combination of controllers, software and/or additional hardware or software can be utilized to selectively energize (make negative) two or more electrodes transmitting charge of the same electrical signal.

To this end, the exemplary circuitry 24 of FIG. 2 includes a series of field effect transistors (FETs) 25a, 25b, 27a, 27b, 29a, 29b, 31a and 31b and capacitors 32 configured to apply a common signal to a musculature through a plurality of electrodes 20a-20d. The transistors allow the circuitry 24 to selectively energize electrodes 20a-d in response to input from NAND gates 34. A counter 36 further affects and coordinates operation of the NAND gates 34, which work in cooperation with FETs 25a, 27a, 29a and 31a to broker voltage from a positive voltage source 38. For purposes of the embodiment, a suitable positive voltage source 38 may comprise a battery, but can alternatively or in addition include any conventional source of voltage. Further features of the circuitry 24 can comprise frequency and pulse width controls 42, 44, as well as a pulse generator 43, voltage level control 40 and rest period hardware 50. Another aspect of the circuitry 24 may include shape controls 39 configured to affect the shape of a pulse or other waveform.

In operation, a pair of FETs 25a,b may be associated with the output of a single electrode 20a of the plurality of electrodes 20a-20d. A second set of transistors 27a,b may control output to a second electrode 20b; a third set 29a,b controls electrode 20c, and transistors 31a and 31b direct the operation of a binary signal to a fourth electrode 20d. In this manner, the transistors 25a, 25b, 27a, 27b, 29a, 29b, 31a and 31b work in pairs to broker voltage from either the negative ground or the positive voltage source 38 to the skin of the user via their respective electrodes 20a-d.

In one sense, a transistor pair 25a,b functions as a gate or an analogous mechanism configured to selectively pass voltage from the source 38/voltage level control 40 to an applicable electrode 20a-d or from the negative ground 52. To this end, it should be understood by one skilled in the art that their functionality could be supplanted by a microprocessor, sequence of switches, other transistors or alternative circuitry optimized to pass voltage to an electrode in a manner consistent with the principles of the present invention. Of note, the circuitry 24 may incorporate capacitors 32 between the transistors of higher potential 25a, 27a, 29a, 31a and the NAND gates 34 to prevent charge from the source 38 from bleeding into and subsequently damaging the NAND gates 34. The capacitors 32 may further function to disable a respective FET by returning the gate of the FET to positive.

An electrode 20a may remain positive so long as the NAND gates 34 sustain a binary low signal to transistor pair 25a,b. Conversely, two or more electrodes 20b, 20c, and 20d may remain negative so long as the NAND gates 34 sustain binary high signals to transistor pairs 27a,b, 29a,b, and 31 a,b. More particularly, when signal protocol calls for the electrode 20a to transmit a positive signal, a low binary signal arrives from the NAND gates 34 at transistor pair 25a,b. The absence of the high binary signal causes the transistor 25b to turn off. As such, the transistor 25a turns on, connecting the electrode 20a to the voltage level control 40. The NAND gates 34 may thus manipulate any of the transistors 25a, 25b, 27a, 27b, 29a, 29b, 31a and 31b according to signal protocol. Such protocol typically calls for three electrodes to be negative, while the fourth is allowed to produce the positive signal. Among other benefits, this arrangement distributes the negative charge associated with pain upon transmission.

The graph of FIG. 3 shows respective discrete counter outputs 301-304 for the electrodes 20a-d of FIG. 2 in accordance with such an application. The counter outputs 301-304 are shown as functions of time, t. Thus, the exemplary time increments t=1 through t=5 of FIG. 3 may correspond to periods within which the signal wave forms may appear in accordance with counter 36 assignments. For example, the counter 36 of FIG. 2 may enable a time slot 307 of FIG. 3 for electrode 20a for the period spanning t=1 to t=2. As such, the electrode 20a is able to transmit positive signal waveforms for the duration of the time slot 307. Conversely, the counter 36 may prevent the same electrode 20a from becoming positive during the subsequent periods of time from t=2 to t=5. During these times counter 36 output 37a remains low and the other counter 36 outputs 37b, 37c and 37d take their turns to allow their respective outputs to go positive. At time t=5 the counter output 37a transitions to positive again allowing electrode 20a to produce a positive signal once again from time slot t=5 to t=6, starting the cycle all over again.

At time t=0 of FIG. 3, respective time slots 307 of all four electrodes 20a-d are enabled by the counter 36, and all the electrodes may be negative as may be dictated by signal protocol. As the electrodes 20a-d are at the same potential, the common signal has no where to drain, and consequently, none of the electrodes 20a-d may apply the signal to the skin of a user. During time t=1, a signal may be inserted in accordance with signal protocol and electrode 20a may transition to positive, while the three other electrodes 20b-d may remain negative. That is, electrodes 20b-d, being grouped to ground, actively transmit the negative aspect of the waveform into time space 307 from t1 to t2, while a fourth electrode 20a transmits the positive counterpart of the signal, for the same period, t=1-2. Thus, the signal transmitted from the negative electrodes 20b-d drains through the body into the positive electrode 20d.

During the period spanning t=2 to t=3, one of the previously negative electrodes 20b may become positive, while electrode 20a transitions to negative. At time t=3, a different combination of negative electrodes 20a, b and d may transmit the signal to a user and into now positive electrode 20c. Of note, such a firing scheme is enabled by the time slots prescribed by the counter 36. At time t=4, electrode 20d may transition to positive while the other electrodes 20a, b and c are negative. The firing sequence may repeat at t=5 with electrode 20a returning to positive status.

While the firing sequence illustrated in FIG. 3 may be executed in accordance with the underlying principles of the present invention, it should be appreciated that other transistor configurations and software firing schemes may be arranged to realize different effects in a manner remaining consistent with embodiments of the invention. For instance, two negative electrodes 20a and 20b may transmit the same signal to one positive electrode 20c on the same circuit. In another discrete circuitry embodiment such as shown in FIG. 1, the transistors 25a, 25b, 27a, 27b, 29a, 29b, 31a and 31b may be alternatively cued such that a single negative electrode 20a drains into multiple positive electrodes 20b-d. Such an arrangement may be used to appropriate where a user wishes to direct the path of the signal's exodus through multiple positive electrodes. In the software embodiment of FIG. 1, certain electrode(s) 20a may be selectively turned off, or neutral, while others 20b-d continue to function in either positive or negative mode. As such, a neutral electrode 20a will not conduct a charge for the period it remains neutral. Moreover, the neutral assignments of electrodes may vary in manner over the course of a stimulating session, to include the rotational fashion discussed above in the context of positive firing assignments. As such, an electrode 20a centered in relation to remaining electrodes 20b-d may transition to neutral in accordance with a preprogrammed firing scheme to affect the path traveled throughout the musculature by the signal. Such a feature may additionally have application in treating or masking pain. In any case, the formerly positive electrode 20a may subsequently resume its positive or negative status according to signal protocol, while one or more electrodes 20b may transition to neutral for a next interval. In this manner, the flexible electrode firing configuration of the present invention enables any combination of electrodes 20a-d to selectively and simultaneously transmit any known signal at any given point in time, t, of a developmental/therapeutic session.

Furthermore, it should be appreciated that other hardware, to include other types of transistors, as well as software and microchip implementations can be substituted for the FETs 25a, 25b, 27a, 27b, 29a, 29b, 31a and 31b in accordance with the principals of the present invention. The electrodes 20a-d may be optimally arranged according to the size, sensitivity and other properties of the musculature, as well as in any manner that accounts for characteristics of the stimulating signal. For instance, electrodes may be dispersed more widely when stimulating a large musculature in order to cover its associated dimensions. For that matter, more or less electrodes may be employed for a given application depending on the desired stimulating effects. A user may place electrodes at strategic points along the musculature to affect the particular tissues of the musculature located at that position to a greater extent than a more sensitive or less therapeutically critical grouping of tissues. For example, denervated tissues of a musculature may programmatically receive higher contractile signals than healthy portions of the musculature, which could experience pain under the high charges. The same tissues could then be subjected to proportional reversing currents in order to maintain zero net charge through the course of a treatment session.

Similarly, the number, timing and type of stimulating signals employed in a single application may be varied according to therapeutic or developmental protocol. Notably, such signal applications may be coordinated with the electrode configuration to selectively stimulate a musculature. In this manner, an embodiment can support multiple levels of precision control and signal/delivery variation unavailable to conventional stimulation machinery. Program code may alter the signal and electrode settings and/or sequences to account for muscle balance, accommodation, nutritional restoration, pain tolerance, zero net balance, or even imbalance to cause controlled chemical changes in the body.

Such signal protocol may be reflected in the operation of the NAND gates 34 of FIG. 2. The NAND gates 34 can sequentially or otherwise selectively transmit binary signals from its ports 35a-d in accordance with the firing assignments dictated by signal protocol. Such protocol can account for which electrode 20a-d is to be negative for a given sequence, and may thus indirectly account for the operation of corresponding transistors 25b, 27b, 29b, 31b. Thus, the transistors 25b, 27b, 29b, 31b marry operation of the electrodes 20a-d to the NAND gates 34 and associated signal protocol.

The NAND gates 34, in turn, may receive pulses from generator 43 while taking further direction from a decade counter 36 with discrete outputs or other counting mechanism. One embodiment may configure such a counter 36 to output a signal from ports 37a-d in sequential fashion. Namely, the counter 36 may transmit a first signal from a first port 37a. A subsequent signal from the counter 36 may originate from port 37b, followed by 37c, and so on, until the counter 36 cycles back to port 37a.

Output from the ports 37a-d may affect operation of the NAND gates 34. Namely, the ports 37a-d of the counter 36 may be hardwired into the NAND gates 34 such that output from different ports 37a-d of the counter 36 into respective input ports 33a-d of the NAND gates 34 cause the NAND gates 34 to transmit a binary signal(s) to a different transistors 25b, 27b, 29b, 31b. For instance, activation of port 37a may allow the NAND gates 34 to send a negative binary signal to transistors 25a,b, causing electrode 20a to be positive. The counter 36 may simultaneously emit another signal from ports 37b-d that causes the NAND gates 34 to send a positive binary signal to the other transistors 27a,b, 29a,b, 31a,b, causing corresponding electrodes 20b-d to become negative.

In one configuration, the counter 36 may thus cycle through the ports 37a-d to affect the firing of the electrodes to vary the respective roles of the electrodes 20a-d. For instance, one electrode 20d of the four 20a-20d may initially be positive, while the others 20a-c are negative. A subsequent signal or portion of a signal may then drain through a new positive electrode 20a, while the formerly positive electrode 20d actively transmits along with the remaining electrodes 20b,c. To this end, the counter 36 may incorporate or wire into reset 30 controls that cause the counter 36 to re-sequence through its respective ports 37a-d after a proscribed routine or in response to input corresponding to the last electrode 20d of a firing sequence.

As such, the reset 30 may cause the counter 36 to repeat transmission from a first port 37a in response to detecting a signal emitted from the last port 37d of a given sequence. Such a routine may correlate to that of a profile retrieved from the database 1 5 of FIG. 1 if a programable controller 1 6 is used in place of the discrete components/circuitry 24 illustrated in FIG. 2. Moreover, one skilled in the art will recognize that more paths/ports 37a-d of the counter 36 may be employed where greater numbers of channels are desired. Similarly, more output ports from the NAND gates 34 may be utilized where more channels are desired.

Significantly, the flexibility enabled by the counter 36 and gate 34 cooperation relieve a great measure of the signal repetition conventionally affecting tissues of a musculature proximate an electrode 20a. Not only can the charge of an incoming signal be distributed among other negative electrodes 20b-d, but those same electrodes 20b-d may sequentially revert to positive. Interjecting periods of positive mode operation for an electrode 20a may provide polar recovery for the tissues proximate the electrode 20a. This feature may further provide tissues an opportunity to uniformly consume nutrition accumulated during the relatively low level. Introduction of this period may help to combat accommodation. Of note, while the counter 36 and gates 34 function in accordance with the principles of the present invention, it should be appreciated that they could be supplanted by other suitable hardware, to include one or more microprocessors/controllers 1 6 as shown in FIG. 1.

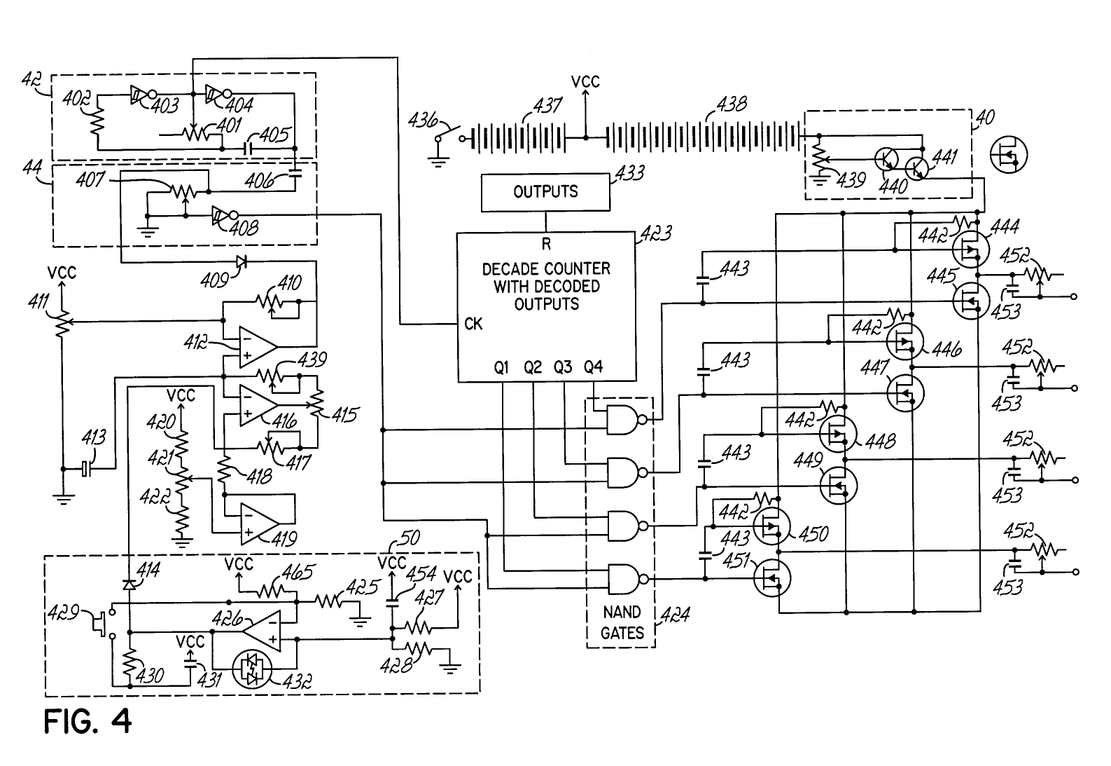

As should be appreciated by one skilled in the art, control of parameters impacted by blocks 39, 42 and 44 may be realized by a suitable interface, such as that shown at block 11 of FIG. 1. For instance, an operator may utilize such an interface 11 to set the frequency at which the counter 36 of FIG. 2 may operate. As such, controls of the interface 11 of FIG. 1 may dial directly into the hardware and/or software comprising block 42 as described below in greater detail. The functionality of block 42 may control or otherwise affect a clock mechanism 45 or other speed setting equivalent. A suitable clock 45 may be configured to determine the frequency of the waveforms conveyed within the signal. One skilled in the art can appreciate that such a clock 45 structure could be constructed according to any number of digital and analog designs, to include combinations of resistors and capacitors, or it may be programmed into a controller 1 6 as shown in FIG. 1. For instance, dashed block 42 of FIG. 4 includes one exemplary embodiment of a clock mechanism comprising resistors 401, 402, Schmitt triggers 403, 404 and capacitor 405 that is suited for application with the hardware environment of FIG. 2. As such, controls at block 42 of FIG. 2 may dictate the rate at which pulses arrive at electrodes 20a-c. This rate control stems from the series connected relationship of the output of block 42 to that of the counter 36 and gate 34. More particularly, the frequency set at block 42 determines the frequency at which the NAND gates 34 and counter 36 are prompted to output their signals. Those signals, in turn, determine the frequency at which charge from the source 38 is applied to the user in form of the signal.

Similarly, an operator may preprogram or otherwise select widths of waveforms at block 44. As above, the waveform characteristic may be selected at an interface 11 such as is included in FIG. 1. As such, suitable signals may be set according to signal protocol or user tolerance and goal specifications. For instance, a user may configure pulses of a signal to be around 200 microseconds in length. Other signals compatible with the embodiment may alter the width as between successive pulses or signal applications. One skilled in the art can appreciate that circuitry suitable to realize the functionality of block 44 may be accomplished in a number of ways in accordance with the principles of the present invention. For instance, block 44 of FIGS. 2 and 4 may use a resistor 405, Schmitt trigger 406 and diode 407 determine pulse width.

In one embodiment, the duration of the signal leaving block 44 is carried over or otherwise proportionally reflected in the binary signal leaving the NAND gates 34. The duration of the binary signal from the applicable output port 37a of the counter 36 determines the maximum possible length of the binary signal emitted from the NAND gates 34 to the transistors 25a, 25b, 27a, 27b, 29a, 29b, 31a and 31b, while the pulse generator 43 dictates the signal length, itself. As discussed above, the absence of a positive binary signal at the transistors 25a, 25b, 27a, 27b, 29a, 29b, 31a or 31b can cause the charge from the source 38 to be channeled to respective electrodes 20a-d for the duration of the binary signal. Thus, the causal relationship of block 44, the pulse generator 43, the NAND gates 34 and the transistor pair 25a, 25b, 27a, 27b, 29a, 29b, 31a and 31b may function to determine the duration of the charge or signal applied to the user according to those settings established at block 44.

The magnitude of that charge is configurable at block 40 of FIG. 2. Block 40, which may comprise a series of interconnected transistors 440, 441 as shown in block 40 of FIG. 4, can affect the amount of voltage delivered to the user in the signal waveform. Thus, block 40 of FIG. 2 may allow the aggregate charge to be adjusted for user tolerances and developmental goals. Of note, the distributed application of the aggregate charge allows for pain normally associated with comparable voltage levels in conventional applications to be apportioned. Consequently, greater penetration is achieved in accordance with the principles of the present invention. Moreover, the aggregate voltage may be proportionately adjusted at block 40 to accomplish greater penetration with less pain than that associated with lesser penetration using conventional applications. Furthermore, the circuitry 24 can actually enhance, and where desired, supplant, the restorative purpose of rest periods embedded within a signal by providing relief to tissues on other developmental levels. Thus, rest can be provided independent of block 50 and signal protocol on a sarcomere level by virtue of the selective nature of the electrode firing configuration of the embodiment.

In addition to rest intervals, the same principles afforded by the selective firing feature of the present embodiment can apply equally to other characteristics of conventional signals. Such signal characteristics include resonant pulses as disclosed in U.S. application Ser. No. 10/0447,745, entitled Resonant Muscle Stimulator, which was filed by inventor James Campos on Jan. 15, 2002 and is hereby incorporated by reference in its entirety, as is U.S. Pat. No. 5,097,833, entitled Transcutaneous Electrical Nerve and/or Muscle Stimulator, which was filed by the same inventor on Sep. 19, 1989. Operators embed such characteristics into known signal sequences to realize specific advantages with relation to a nerve, muscle and circulatory tissues. An embodiment of the invention preserves the optimized sequence, amplitude, frequency and other characteristics of the signal. Moreover, their cumulative effects are enhanced by the selective electrode 20a-d firing sequence of the present embodiment such that conventional limitations associated with nutritional starvation, accommodation, pain tolerance, as well as polar and developmental unbalance can be further reduced.

In the absence of the selective transmission feature of the present invention, the tissues proximate the negative electrode can become nutritionally depleted as the train of pulses continues over time, eventually succumbing to accommodation and other detrimental effects. Moreover, the sarcomeres of the tissues may react to a lesser extent to each subsequent pulse as they accommodate or otherwise become accustomed to the pulses presented to localized tissues of a musculature. Similarly, tissues along the path of travel of the signal throughout the musculature react less to a subsequent pulse. Sensory nerves along the path of the signal can become sensitized over time, inducing painful stinging in response to repeated pulses.

An embodiment of the present invention can overcome such limitations by affecting one of the major factors plaguing conventional applications. Namely, the embodiment of FIGS. 1 and 2 can reduce detrimental affects incurred as a function of repetition relative to the position of the negative 20a electrode on a musculature. The circuitry 24 can break up repetition not only by transmitting the signal from different electrodes, but by further transmitting different portions of the signal to different electrodes over the course of a stimulating session. Thus, tissues of a musculature proximate a given electrode 20a can experience variation in both signal characteristics and the frequency of the signal's arrival. Unlike a prior art signal that is repetitively transmitted from a single electrode for an entire application, the circuitry 24 of FIG. 2 mitigates accommodation, starvation and pain by distributing the signal over different positions proximate the musculature during the course of a single stimulating signal and/or session. That is, portions of the signal can be selectively transmitted to different electrodes to realize effects specific to particular tissue locations along the musculature.

A conventional signal can be applied to the musculature through preselected, alternating points of entry and exit corresponding to alternating electrode 20a-d assignments. Accordingly, signal paths traveled by the signal through the musculature will vary as per selected points of entry/exit. This variation mitigates the effects of accommodation, tetany, nutritional depletion and polar imbalance by effectively distributing the cause of the detrimental effects over larger areas of the musculature and larger increments of time. As such, no single portion of the muscle becomes desensitized as quickly or as much to the pulses. In this manner, the distributed and selective nature of the electrode firing configuration 20ad can enhance signal effectiveness by decreasing the incidences of repetition and charge concentration.

To this end, profiles may be established that determine firing sequences for different applications. While such profiles may be hardwired into circuitry using transistor settings of the NAND gates 34 and/or counter 36, another embodiment may store the profiles in the database 1 5 of FIG. 1 such that the controller 1 6 may retrieve and process the profile to realize the associated firing protocol. One such stored profile may call for each consecutive pulse of a signal to be transmitted to a different electrode 20a-d. Another profile may require several consecutive pulses to be applied to the user via the same electrode 20a before sending a subsequent pulse sequence to another electrode 20b. Other profiles may cause the electrodes 20a-d to fire non-consecutively or disproportionally relative to one another. Such a profile may be appropriate where certain tissues proximate an electrode 20a firing with greater relative frequency requires proportionally greater stimulation relative to other tissues of the musculature. Still other profiles may direct pulses of a signal having a particular characteristic, such as pulse length or high voltage, to the same or different electrode(s) 20a over the course of a stimulating session.

Thus, the selectivity of the circuitry 24 of FIG. 2 can further enable more precise stimulation of certain areas of a musculature that must be particularly developed. For instance, a specific tissue area of a musculature in need of blood flow may be targeted to receive longer pulses by programming the stimulator to send them to an electrode 20a proximate the targeted area. This level of control effectively allows users to program a stimulating signal protocol on an additional layer. That is, an embodiment of the present invention still accommodates variation in signal characteristics useful in overcoming pain, starvation, polar extremes and accommodation, but additionally acts to further break up repetition, over stimulation and other causes of effects detrimental to stimulation. In this manner, the musculature still receives the aggregate benefits associated with an optimized stimulating signal regime, only now the delivery of the signal is accomplished in a less disruptive manner via a plurality of coordinated, negative electrodes. Thus, the detrimental effects that conventionally accompany such signals are proportionately distributed so that no single sensory nerve becomes agitated.

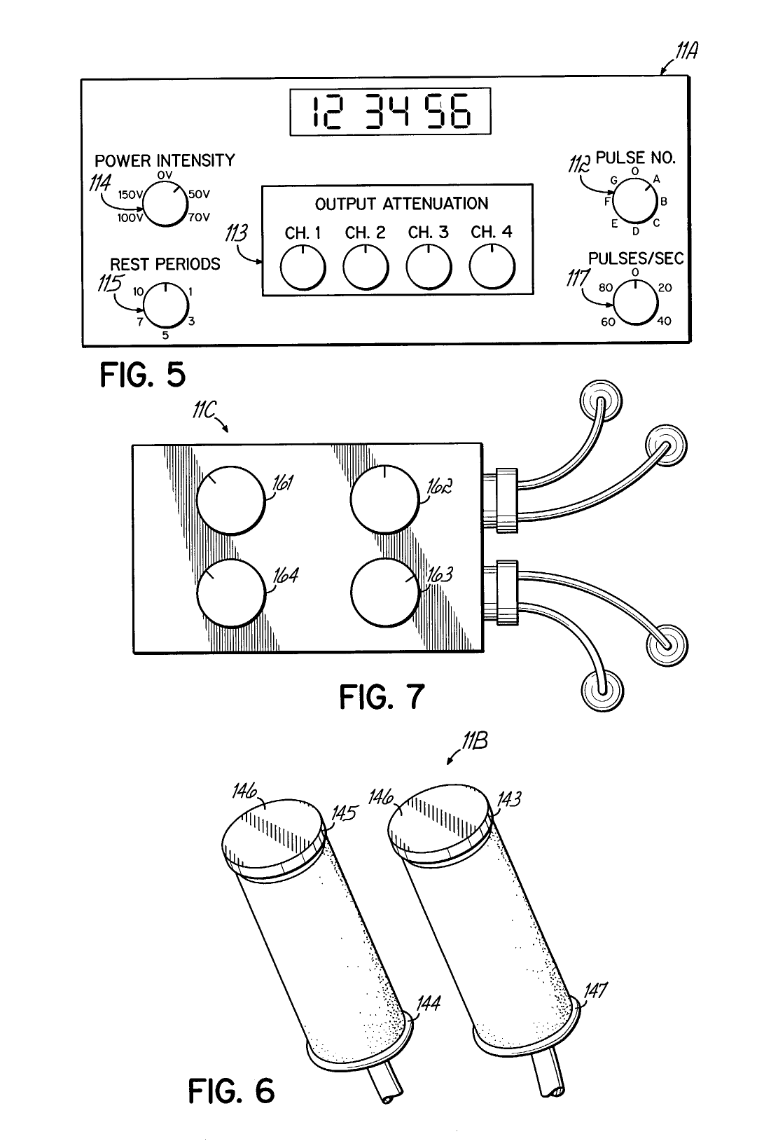

The exemplary interfaces 11A-C of FIGS. 3-5 enable a user/operator to adjust signal characteristics associated with the signal, as well as to determine from which electrode 20a-d and at what time a portion of the signal will be transmitted. To this end, suitable interfaces 11A-C shown in FIGS. 3-5 may include controls that correspond to the functionality of blocks 34, 36, 42 44 and 46 of FIG. 2. To this end, the interface 11 A of FIG. 5 includes settings relating to pulse rate 11 7, amplitude 11 4, individual channel output attenuation for balance 11 3, rest 11 5 and distribution profile 11 2. More particularly, the user may manually adjust voltage/intensity using dial 11 4 of the interface 11 A to a setting that typically ranges from about 1 to about 1 50 volts. As such, corresponding block 40 of FIG. 2 would be accordingly adjusted to realize the selected voltage setting. Of note, the above stated range is merely exemplary and increased voltage levels may be possible with less perceived pain due to the distributed signal feature of the embodiment. Substantially higher voltages may be necessary in special therapeutic cases, such as with a denervated patient.

An operator may adjust another dial 11 7 of the interface 11 A to determine the frequency of pulses associated with the signal. Most treatment applications typically require anywhere from about 25 pulses (or pulse groups) per second to about 70 pulses/groups per second. Other dials 11 3 may proportionally control the final intensity or voltage output associated with a signal relative to each other. When applicable, an operator may adjust rest periods via dials 11 5. A user may select a profile, or electrode firing sequence, using dial 11 2 of the interface 11A.

The dial 11 2 may include multiple settings, each setting initiating a software/hardware electrode firing sequence for the applied signal. For instance, a first setting of the dial 11 2 may correlate to a profile that causes three electrodes 20a-c of FIG. 2 to be initially negative, while the fourth electrode 20d is held positive. The applicable profile may then initiate a sequence where each negative electrode consecutively takes a turn at being positive for a preset signal increment until the profile cycles back to the original positive electrode 20d. The profile may then call for the same firing sequence to repeat, or may segue into another firing rotation.

Another purpose for dial 11 2 may be to select the number of pulses that are generated in each time window from t=1 through t=5 for a simple, mathematically uniform distribution of the signal. Another setting of dial 112 may call for every third pulse or pulse grouping of a signal to be transmitted to a particular electrode 20b. A third profile setting may transmit three consecutive pulses of a signal to negative electrodes 20b,d, then send the subsequent two pulses to another grouping of electrodes 20a,c,d. One skilled in the art can appreciate that the present embodiment can accommodate any number of firing schemes to suit a desired developmental/rehabilitative regime, to include a quasi-random firing effectuated by a suitable program.

As such, input received from the user interface 11 may relate to voltage intensity, pulse rate, pulse duration, charge balance, phasic modulation, rest periods, profile selection and firing assignment, among others. Furthermore, while the interface 11A of FIG. 3 receives input from a series of dials, any combination of switches, keyboard, touch screen/pad, buttons, modem, microphone, or other known input mechanism may alternatively be employed. Alternatively or in addition, a suitable user interface 11 of FIG. 1 may place little or no physical demands on a user. For instance, a suitable interface 11 may include voice recognition software, or incorporate handles or pedals that may be manipulated by merely bumping or squeezing.

A pair of such handles 146 comprise the exemplary interface 11 B of FIG. 6. A user may grip orient, or contact the handles 146 in such a manner as to affect the voltage, frequency, distribution sequences and rest periods associated with signals. For instance, a user may adjust pulse rate by tapping opposite ends 143 and 144 of the handles 146 together. Another embodiment may interpret the same action as a request from the user to incrementally increase voltage. Conversely, tapping opposite ends 145 and 147 may cause a decrease in voltage. Contacting another pair of ends 143, 145 of the handles 146 may initiate a period of rest for the user, temporarily halting transmission of the stimulating signal. Other parameters, such as profile selection, may be accessible to the user by contacting respective bottoms 144, 147 of the handles 146 together. Such contact may alternatively change a mode of the application, altering command/contact sequences of the handles 146 to allow for the adjustment of additional parameters.

Another embodiment illustrated in FIG. 7, shows a battery operated user interface 11 C configured to fit within a pocket or otherwise attach to the clothing of a user. As with the larger, stationary embodiment shown in FIG. 5, the user interface 11 C of FIG. 7 incorporates multiple dials 161 -164 with which the wearer may adjust stimulator settings. More particularly, dial 1 61 may communicate required voltage levels to the stimulator, typically ranging from a fraction of 1 volt to about 1 50 volts. Dial 1 62 may adjust the firing sequence/profile of the electrodes as discussed above. Dial 1 63 of the interface 11C may control the frequency of waveforms generated by the stimulator, and dial 1 64 may interject a preset ratio of rest periods between pulses/waveforms.

A user interface 11 of another embodiment may incorporate a hysteresis loop and sensors configured to monitor contractile, diagnostic or other patient/user reactions. Programming in communication with the interface 11 may automatically initiate adjustment of the signal in accordance with presented feedback. For instance, a sensor monitoring the heart rate of a patient may cause the stimulator 1 2 of FIG. 1 to step down voltage or interject a rest period in response to detecting an elevated heart rate. Moreover, a suitable user interface 11 may enable both the user and the operator to access the interface. As such, the feature allows an athlete or patient to adjust signal charge and other parameters of the stimulator signal per their own tolerance levels and unique fitness goals.

Also of note, the flexible interface 11 and electrode configuration of the above described embodiment may enable an athlete or patient to perform athletic or therapeutic motions while the stimulator concurrently exercises the musculature. As such, an operator may limit the number of electrodes used in an application to accommodate a desired range of motion. As such, a user may simulate an arm swing appropriate for a tennis racket, baseball bat or golf club while electrodes on the swinging arm communicate muscle building signals. This feature enables a musculature of the athlete to be stimulated at different stages of contraction, translating into more balanced muscle development and training.

It will be appreciated that the generation of the signals and their application discussed herein may be implemented using hardware and/or software to store and/or generate the appropriate profiles and shapes, and that such implementations would be within the abilities of one of ordinary skill in the art having the benefit of this disclosure. Moreover, one skilled in the art can appreciate that circuitry suitable to realize the functionality of the circuitry 24 included in FIG. 2 may be accomplished in a number of ways in accordance with the principles of the present invention.

One skilled in the art will recognize that the functions of the counter 36, gate 34 and transistors 25a, 25b, 27a, 27b, 29a, 29b, 31a and 31b could alternatively be achieved by software and/or other hardware components. Furthermore, any of the hardware comprising the blocks shown in FIG. 2 could be augmented with additional equipment that is conventionally arranged to realize greater efficiency and accuracy. For instance, a series of optical amplifiers with feedback loops could be hardwired in such a way as to provide hysteresis. Such hysteresis could decrease the effects of variance in electrical properties present in the FETs 25a, 25b, 27a, 27b, 29a, 29b, 31a and 31b. Moreover, other controls not shown in FIG. 2 may be included for the purpose of altering characteristics of the signal(s) or its associated waveforms.

Furthermore, while the invention has been described in the context of a stimulator, controller, computer or other processor, those skilled in the art will appreciate that the various embodiments of the invention are capable of being distributed as a program product in a variety of forms, and that the invention applies equally regardless of the particular type of signal bearing medium used to actually carry out the distribution. Examples of signal bearing media include but are not limited to recordable type media such as volatile and non-volatile memory devices, floppy and other removable disks, hard disk drives, magnetic tape, optical disks (e.g., CD-ROMs, DVDs, etc.), among others, and transmission type media such as digital and analog communication links.

While the present invention has been illustrated by a description of various embodiments and while these embodiments have been described in considerable detail, it is not the intention of the applicants to restrict or in any way limit the scope of the appended claims to such detail. Additional advantages and modifications will readily appear to those skilled in the art. For instance, while an electrode configuration of one embodiment may call for multiple negative electrodes to fire a signal that will drain into a single positive electrode, other applications consistent with the principles of the present invention may require a signal to drain into a plurality of positive electrodes. Such a configuration may facilitate desired signal paths throughout a musculature. Moreover, the circuitry 1 4 and electrodes 20a-d of FIG. 2 can cooperate to function in a manner analogous to a voltage divider upon the signal's exodus.

The invention in its broader aspects is therefore not limited to the specific details, representative apparatus and method, and illustrative example shown and described. Accordingly, departures may be made from such details without departing from the spirit or scope of applicant's general inventive concept.

What is claimed is:

1. A method of stimulating a musculature with a plurality of electrodes proximate the musculature, comprising applying a signal to the musculature via at least two electrodes of the plurality of electrodes.

2. The method of claim 1, further comprising allowing the signal to exit the musculature through at least a third electrode of the plurality of electrodes.

3. The method of claim 1, wherein applying the signal across the musculature via the at least two electrodes of the plurality of electrodes further includes:
selectively applying a first portion of the signal to the musculature via a first electrode of the at least two electrodes during a first time period; and
selectively transmitting a subsequent portion of the signal to a second electrode of the at least two electrodes.

4. The method according to claim 3, wherein selectively applying the first and subsequent portions of the signal includes accessing from a memory a profile having firing assignments, wherein the firing assignments determine what portion of the signal is sent to each of the at least two electrodes for the first and subsequent time periods.

5. The method according to claim 1, wherein the signal has different waveform characteristics.

6. The method according to claim 5, wherein the different waveform characteristics are selected from among a group comprising: frequency, width, amplitude, spacing, polarity, shape and some combination thereof.

7. The method according to claim 1, further comprising generating the signal.

8. The method according to claim 7, wherein generating the signal includes generating the signal in response to input from a user interface.

9. The method according to claim 8, wherein the input received from the user interface concerns a parameter selected from a group consisting of: voltage intensity, pulse rate, pulse duration, charge balance, phasic modulation, rest periods, profile selection, firing assignment and some combination, thereof.

10. The method according to claim 8, further comprising configuring the user interface to attach to a wearer.

11. The method according to claim 8, wherein the user interface fits within a pocket of the wearer.

12. The method according to claim 8, wherein the user interface transmits the input in response to commands received from a source selected from a group consisting of: a handle, pedal, dial, button, switch, voice recognition software, diagnostic equipment, motion sensor and some combination, thereof.

13. The method according to claim 1, further comprising applying at least one additional signal to a user through at least two distinct electrodes of the plurality of electrodes.

14. The method according to claim 1, wherein applying the signal across the musculature via at least two electrodes of the plurality of electrodes further includes defining multiple points of entry for the signal at the at least two electrodes.

15. The method according to claim 1, wherein the signal exiting the musculature further includes defining multiple points of exit at a portion of the plurality of electrodes.

16. The method according to claim 1, further comprising transitioning at least a fourth electrode of the plurality of electrodes to neutral.

17. A method of stimulating a musculature using a plurality of electrodes proximate the musculature, comprising:
selectively applying a first portion of a signal to the musculature via a first electrode of the plurality of electrodes during a first time period; and
selectively transmitting a second portion of the signal to a second electrode of the plurality of electrodes.

18. The method according to claim 17, wherein selectively applying the first portion of the signal further includes simultaneously applying the first portion of the signal via another electrode.

19. The method according to claim 17, wherein selectively applying the first and subsequent portions of the signal includes accessing from a memory a profile having firing assignments, wherein the firing assignments determine what portion of the signal is sent to each of the at least two electrodes for the first and subsequent time periods.

20. The method according to claim 17, wherein the signal has different waveform characteristics.

21. The method according to claim 20, wherein the different waveform characteristics are selected from among a group comprising: frequency, width, amplitude, spacing, polarity, shape and some combination thereof.

22. The method according to claim 17, further comprising generating the signal.

23. The method according to claim 22, wherein generating the signal includes generating the signal in response to input from a user interface.

24. The method according to claim 23, wherein the input received from the user interface concerns a parameter selected from a group consisting of: voltage intensity, pulse rate, pulse duration, charge balance, phasic modulation, rest periods, profile selection, firing assignment and some combination, thereof.

25. The method according to claim 23, further comprising configuring the user interface to attach to a wearer.

26. The method according to claim 23, wherein the user interface fits within a pocket of the wearer.

27. The method according to claim 23, wherein the user interface transmits the input in response to commands received from a source selected from a group consisting of: a handle, pedal, dial, button, switch, voice recognition software, diagnostic equipment, motion sensor and some combination, thereof.

28. The method according to claim 23, further comprising applying at least one additional signal to a user through at least two distinct electrodes of the plurality of electrodes.

29. A method of stimulating a musculature with a plurality of electrodes proximate the musculature, comprising applying a common signal across the musculature via a first electrode of the plurality of electrodes, wherein the signal exits the musculature through at least a second and a third electrode of the plurality of electrodes.

30. An apparatus for stimulating a musculature, comprising:
a stimulator configured to produce a signal for transcutaneous delivery to a musculature, the stimulator being operable to apply the signal to the musculature via at least two electrodes of a plurality of electrodes.

31. The apparatus of claim 30, wherein the signal exits the musculature through at least a third electrode of the plurality of electrodes.

32. The apparatus of claim 30, wherein the stimulator selectively applies a first portion of the signal to the musculature via a first electrode of the at least two electrodes during a first time period, and selectively transmits a subsequent portion of the signal to a second electrode of the at least two electrodes.

33. The apparatus according to claim 32, wherein the stimulator selectively applies the first and subsequent portions of the signal according to a stored profile having firing assignments, wherein the firing assignments determine what portion of the signal is sent to each of the at least two electrodes for the first and subsequent time periods.

34. The apparatus according to claim 30, wherein the signal has different waveform characteristics.

35. The apparatus according to claim 34, wherein the different waveform characteristics are selected from among a group comprising: frequency, width, amplitude, spacing, polarity, shape and some combination thereof.

36. The apparatus according to claim 30, wherein the stimulator generates the signal in response to input from a user interface.

37. The apparatus according to claim 36, wherein the input received from the user interface concerns a parameter selected from a group consisting of: voltage intensity, pulse rate, pulse duration, charge balance, phasic modulation, rest periods, profile selection, firing assignment and some combination, thereof.

38. The apparatus according to claim 36, wherein the user interface attaches to a wearer.

39. The apparatus according to claim 36, wherein the user interface transmits the input in response to commands received from a source selected from a group consisting of: a handle, pedal, dial, button, switch, voice recognition software, diagnostic equipment, motion sensor and some combination, thereof.

40. The apparatus according to claim 30, wherein at least one additional signal is applied to a user through at least two distinct electrodes of the plurality of electrodes.

41. The apparatus according to claim 30, wherein at least a portion of the plurality of electrodes define multiple points of entry for the signal.

42. The apparatus according to claim 30, wherein at least a portion of the plurality of electrodes define multiple points of exit for the signal.

43. The apparatus according to claim 30, wherein the plurality of electrodes includes at least one neutral electrode.

44. An apparatus for stimulating a musculature, comprising:
a stimulator configured to produce a signal for transcutaneous delivery to a musculature, the stimulator being operable to selectively apply a first portion of a signal to the musculature via a first electrode of a plurality of electrodes during a first time period, and to selectively transmit a second portion of the signal to a second electrode of the plurality of electrodes.

45. The apparatus according to claim 44, wherein the stimulator simultaneously applies the first portion of the signal via another electrode.

46. The apparatus of claim 44, wherein the stimulator selectively applies a first portion of the signal to the musculature via a first electrode of the at least two electrodes during a first time period, and selectively transmits a subsequent portion of the signal to a second electrode of the at least two electrodes.

47. The apparatus according to claim 46, wherein the stimulator selectively applies the first and subsequent portions of the signal according to a stored profile having firing assignments, wherein the firing assignments determine what portion of the signal is sent to each of the at least two electrodes for the first and subsequent time periods.

48. The apparatus according to claim 44, wherein the signal has different waveform characteristics.

49. The apparatus according to claim 44, wherein the stimulator generates the signal in response to input from a user interface.

50. The apparatus according to claim 49, wherein the input received from the user interface concerns a parameter selected from a group consisting of: voltage intensity, pulse rate, pulse duration, charge balance, phasic modulation, rest periods, profile selection, firing assignment and some combination, thereof.

51. The apparatus according to claim 49, wherein the user interface is wearable by a user.

52. The apparatus according to claim 44, wherein at least one additional signal is applied to a user through at least two distinct electrodes of the plurality of electrodes.

53. A program product, comprising:
a program for stimulating a musculature, the program configured to initiate application of a signal for transcutaneous delivery to the musculature via at least two electrodes of a plurality of electrodes, wherein the signal exits the musculature through at least a third electrode of the plurality of electrodes; and
a signal bearing medium bearing the program.

54. The program product of claim 53, wherein the signal bearing medium includes at least one of a recordable medium and a transmission-type medium.

55. A program product, comprising:
a program for stimulating a musculature with a signal, the musculature being positioned between a plurality of electrodes, the program configured to initiate selective application of a first portion of the signal to the musculature via a first electrode of the plurality of electrodes during a first time period, and selectively transmit a second portion of the signal to a second electrode of the plurality of electrodes; and
a signal bearing medium bearing the program.

56. The program product of claim 55, wherein the signal bearing medium includes at least one of a recordable medium and a transmission-type medium.
